(19) United States
(12) Patent Application Publication
(10) Pub. No.: US 2021/0093227 A1
(43) Pub. Date: Apr. 1, 2021

(54) IMAGE PROCESSING SYSTEM AND CONTROL METHOD THEREOF

(71) Applicant: CANON KABUSHIKI KAISHA, Tokyo (JP)

(72) Inventors: Yosato Hitaka, Tokyo (JP); Takuya Kubo, Tokyo (JP)

(21) Appl. No.: 17/032,963

(22) Filed: Sep. 25, 2020

(30) Foreign Application Priority Data
Sep. 26, 2019 (JP) 2019-175334
Sep. 26, 2019 (JP) 2019-175565

(51) Int. Cl. A61B 5/107; A61B 5/00; A61B 5/11; G06T 7/00

(52) U.S. Cl. CPC A61B 5/1076; A61B 5/004; G06T 7/0012; A61B 5/1072; A61B 5/1128

(57) ABSTRACT

An image processing system according to the present invention includes at least one memory and at least one processor which function as: an acquiring unit configured to acquire information on a captured moving image: a detecting unit configured to detect, on a basis of the information acquired by the acquiring unit, an edge point of an affected area in a diameter direction thereof from a locus of light moving inside the affected area; and a providing unit configured to provide information on an outer periphery of the affected area on a basis of a plurality of points detected by the detecting unit.

### BACKGROUND OF THE INVENTION

### Field of the Invention

The present invention relates to a technique of image processing that estimates from an image the size of an affected region of an object.

### Description of the Related Art

At medical and caregiving sites, it is demanded to periodically evaluate a bedsore of a bedsore affected patent, and the size of the bedsore is one index to recognize the degree of bedsore progress. WO 2006/057138 discloses measuring the size of a pocket of the bedsore by inserting a light-emitting unit into the pocket, and putting marks on the skin along the contour of the pocket or reading gradations thereof.

According to the method of WO 2006/057138, the operator must perform processing to put marks on the skin or processing to read gradations thereof in a state of holding the light at a position that forms the contour of the pocket. Therefore, the operator performs the procedure to measure the size of the bedsore while paying attention to not allow the light to deviate from the position, which may increase operational stress.

### SUMMARY OF THE INVENTION

With the foregoing in view, the present invention provides a technique to improve operability when the affected region (e.g. pocket of bedsore) is measured.

An image processing system according to the present invention includes at least one memory and at least one processor which function as:

an acquiring unit configured to acquire information on a captured moving image;

a detecting unit configured to detect, on a basis of the information acquired by the acquiring unit, an edge point of an affected area in a diameter direction thereof from a locus of light moving inside the affected area: and

a providing unit configured to provide information on an outer periphery of the affected area on a basis of a plurality of points detected by the detecting unit.

Further features of the present invention will become apparent from the following description of exemplary embodiments with reference to the attached drawings.

### BRIEF DESCRIPTION OF THE DRAWINGS

FIG. 1A to FIG. 1C are diagrams depicting a bedsore;

FIG. 2 is a diagram depicting measurement of a pocket of a bedsore:

FIG. 3 is a block diagram of an image processing system according to this embodiment:

FIG. 4 is a diagram depicting an object according to this embodiment:

FIG. 5 is a block diagram depicting a configuration of an imaging apparatus according to this embodiment:

FIG. 6 is a block diagram depicting a configuration of an image processing apparatus according to this embodiment;

FIG. 7 is a flow chart depicting an operation of an image processing system according to this embodiment:

FIG. 8 is a diagram depicting a method of calculating an area size according to this embodiment;

FIG. 9 is a diagram depicting a method of superimposing information according to this embodiment:

FIG. 10 is a diagram depicting a moving image according to this embodiment;

FIG. 11 is a diagram depicting a moving image analysis processing according to this embodiment;

FIG. 12 is a diagram depicting a display during the pocket measurement operation according to this embodiment;

FIG. 13 is a diagram depicting a display after the pocket measurement operation according to this embodiment:

FIG. 14A to FIG. 14C are diagrams depicting superimposed images according to this embodiment;

FIG. 15 indicates object information according to this embodiment:

FIG. 16A and FIG. 16B are flow charts depicting the moving image analysis processing according to this embodiment;

FIG. 17 is a diagram depicting an image capturing a bedsore including predetermined markers according to this embodiment;

FIG. 18A to FIG. 18C are diagrams depicting a result of combining a light region according to this embodiment;

FIG. 19 is a flow chart depicting an outer periphery drawing processing according to this embodiment:

FIG. 20 is a flow chart depicting a modification of the moving image analysis processing according to this embodiment: and

FIG. 21A and FIG. 21B are diagrams depicting a UI to delete an unnecessary light region according to this embodiment.

### DESCRIPTION OF THE EMBODIMENTS

Embodiments of the present invention will be described with reference to the drawings. Dimensions, materials, shapes and relative positions of the composing elements described in the following embodiment are arbitrary and can be changed in accordance with the configurations and various conditions of the apparatuses to which the present invention is applied. In each drawing, identical or functionally similar elements are indicated by the same reference sign.

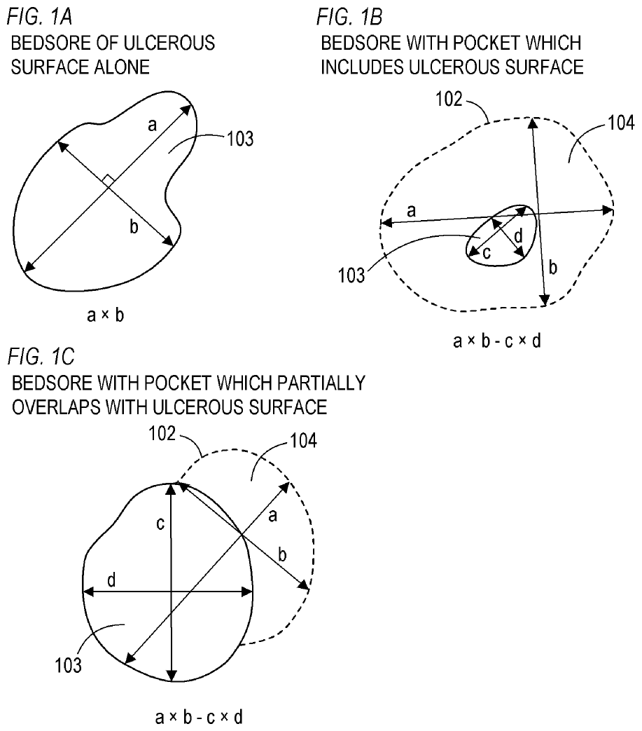

FIG. 1A to FIG. 1C indicate a method of measuring (evaluating) the size of a bedsore. FIG. 1A is an example of measuring the size of only the ulcerous surface of the bedsore. The size of the bedsore is normally determined based on the value that is manually measured by placing a measure on the affected area (ulcerous surface region 103). In concrete terms, the longest direct distance between two points in the ulcerous range of the skin (ulcerous surface region 103) is measured, and this distance is regarded as major axis a of the bedsore. Further, the longest direct distance between two points, that is perpendicular to the major axis a of the affected range of the skin, is measured, and this distance is regarded as minor axis b of the bedsore. Then a value determined by multiplying the major axis a by the minor axis b is regarded as the size of the bedsore. For the other regions as well, the longest direct distance of a region is referred to as the “major axis”, and the longest direct distance that is perpendicular to the major axis is referred to as the “minor axis”.

A typical symptom/classification of a bedsore is a bedsore that has a pocket. The pocket is a cavity that is wider than the affected skin area (ulcerous surface: exposed portion), and in some cases may spread deep and wide under the skin in a portion not visible from the outside (unexposed portion). FIG. 1B and FIG. 1C are examples of a bedsore with a pocket. FIG. 1B is an example of a pocket that encloses an ulcerous surface, that is, a pocket that spreads in all directions from the ulcerous surface, and FIG. 1C is an example of a pocket that partially overlaps with the ulcerous surface, that is, a pocket that spreads in part of the directions from the ulcerous surface. In the case of a bedsore with a pocket, the affected region 102 is the entire region, including the ulcerous surface region 103 and the pocket region 104. To evaluate such a pocket of the bedsore, it is necessary to measure the range where the cavity (pocket) is spread. For example, in the case of a marking method of a pocket using DESIGN-R (R) software, this range is measured by subtracting the size of the ulcerous surface (value determined by multiplying the major axis c and the minor axis d of the ulcerous surface region 103) from a value determined by multiplying the major axis a and the minor axis b of the affected region 102 which includes the ulcerous surface and the pocket.

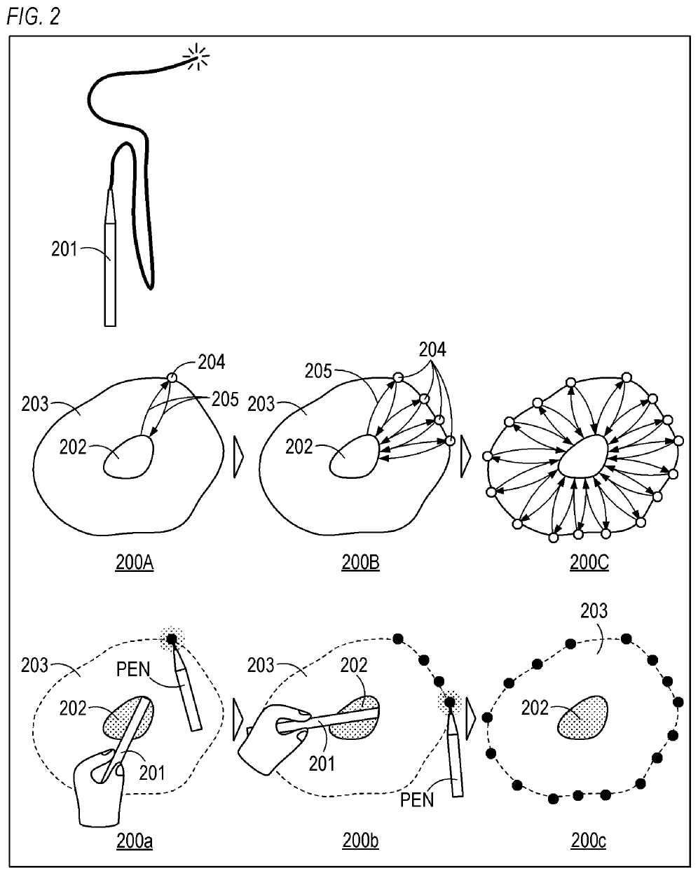

FIG. 2 indicates an overview of a measurement operation to measure a pocket 203 using a light 201. In the measurement operation, the tip (lighting portion) of the light 201 is inserted into the pocket 203 through the ulcerous surface 202. Then the tip of the light 201 is moved toward the edge of the pocket 203, and when the tip of the light 201 reaches the deepest portion (edge of the pocket 203), a position 204 on the skin surface where the light emitted from the light 201 transmits through is marked using a magic marker or the like. Then the light 201 is withdrawn from the pocket 203. The arrow mark 205 indicates the movement of the light 201 at this time. As the arrow mark 205 indicates, the light 201 moves in the diameter direction of the affected area, from a predetermined region near the center of the affected area to the edge of the affected area, and then moves to the predetermined region. This operation is repeated. The states 200A and 200a are states where making is performed on one point, the states 200B and 200b are states where marking is performed on four points, and the states 200C and 200c are states where marking is performed all around the pocket 203. From the plurality of markings all around the pocket 203, the shapes of the outer periphery of the pocket 203 can be determined, and the pocket 203 can be evaluated.

### Embodiment 1

In Embodiment 1, a procedure to measure an area size of the ulcerous surface of the bedsore from a captured image, and create a composite image to measure the size of the pocket region, will be described.

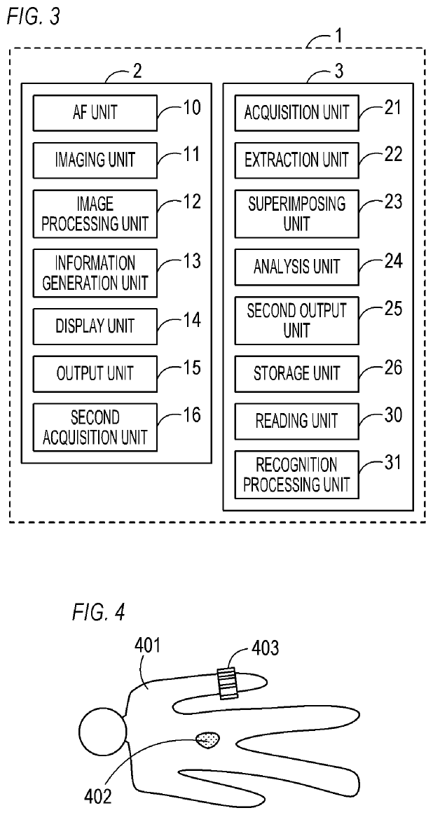

An image processing system according to Embodiment 1 of the present invention will be described with reference to FIG. 3 and FIG. 4. FIG. 3 is a block diagram depicting an example of a functional configuration of the image processing system according to Embodiment 1. The image processing system 1 is constituted of an imaging apparatus 2, which is a portable device, and an image processing apparatus 3. FIG. 4 is a diagram depicting an object that is measured by the image processing system 1. In the description of Embodiment 1, an example of a condition of an affected region 402, generated in the buttocks of the object 401, is referred to as the bedsore.

The image processing system 1 captures an image of the affected region 402 of the object 401, acquires an object distance, extracts an image region corresponding to the affected region 402, detects an outer peripheral shape of the affected region 402, measures the major axis and the minor axis of the affected region 402, and measures the size of the bedsore. Here an area size per pixel may be measured based on the object distance and the angle of view of the imaging apparatus 2, so that the area size of the affected region 402 is measured based on the extraction result of the affected region 402 and the area size per pixel.

In the object 401, a barcode tag 403, on which a one-dimensional barcode (not illustrated) is drawn as the information to identify the object, is attached, so as to link the image data and the ID of the object. The information to identify the object is not limited to a one-dimensional barcode, but may be a two-dimensional barcode (e.g. QR code (R)) or a numeric value. Further, data attached to the information on the ID card (e.g. medical examination card) or an ID number may be used.

The functional configuration of the imaging apparatus 2 will be described. The imaging apparatus 2 functions as an AF unit 10, an imaging unit 11, an image processing unit 12, an information generation unit 13, a display unit 14, an output unit 15 and a second acquisition unit 16.

The AF unit 10 has an automatic focus adjustment function to automatically focus on the object. The AF unit 10 also has a function to output a distance to the object (object distance) based on the moving distance of the focus lens.

The imaging unit 11 captures an image of the object and generates image data of the still image or the moving image.

The image processing unit 12 performs image processing (e.g. development, resizing) on the image acquired by the imaging unit 11.

The information generation unit 13 generates distance information on the distance to the object. For example, the information generation unit 13 generates the distance information based on the distance outputted by the AF unit 10.

The display unit 14 displays an image captured by the imaging unit 11. The display unit 14 also displays information outputted from the image processing apparatus 3 (e.g. information indicating the extraction result of an affected region 402, information on the size of the affected region 402) and the like. Such information may be superimposed and displayed on a captured image. The display unit 14 also displays a composite image that is outputted from the image processing apparatus 3 and that is used for determining the size of the pocket region. The method of creating the composite image will be described later.

The output unit 15 outputs the image data and the distance information to an external apparatus, such as an image processing apparatus 3. The image data is, for example: image data capturing an affected area of the object 401, image data on the object 401 in general, image data capturing such identification information as a one-dimensional barcode drawn on the barcode tag 403, and moving image data during measurement operation using a light.

The second acquisition unit 16 acquires images and evaluation information which indicates a result of evaluating the ulcerous surface region and pocket region, for example, from such an external apparatus as the image processing apparatus 3.

The functional configuration of the image processing apparatus 3 will be described next. The image processing apparatus 3 functions as an acquisition unit 21, an extraction unit 22, a superimposing unit 23, an analysis unit 24, a second output unit 25 and a storage unit 26.

The acquisition unit 21 acquires the image data and the distance information (object distance) outputted by the imaging apparatus 2.

The extraction unit 22 extracts an image region corresponding to the affected region 402 from an image capturing the affected region 402 (image data outputted by the imaging apparatus 2). Extracting a region from an image is referred to as region extraction or region division.

The analysis unit 24 analyzes the information on the size of the affected region 402 extracted by the extraction unit 22 based on the distance information (object distance) generated by the information generation unit 13. Furthermore, the analysis unit 24 analyzes a moving image during the measurement operation using a light, in order to create a composite image to identify a size of the pocket region.

The superimposing unit 23 superimposes information indicating the extraction result of the affected region 402, information on the size of the affected region 402 or the like on the image corresponding to the image data that is used for extracting the affected region 402.

The second output unit 25 outputs the information indicating the affected region 402 extracted by the extraction unit 22, information on the size of the affected region 402 analyzed by the analysis unit 24, the image data acquired by the superimposing unit 23 (image on which information is superimposed) or the like to such an external apparatus as the imaging apparatus 2. The second output unit 25 can also output a composite image, to detect a size of the pocket region, to an external apparatus.

The reading unit 30 reads a one-dimensional barcode (not illustrated) drawn on the barcode tag 403 from the image capturing the barcode tag 403, and acquires the identification information (e.g. object ID) to identify the object 401. The target that is read by the reading unit 30 may be a two-dimensional code (e.g. QR code), numeric value or text.

The recognition processing unit 31 collates the object ID (identification information) read by the reading unit 30 with a object ID that is registered in advance, and acquires the name of the object 401.

The storage unit 26 generates records based on an image capturing the affected region 402 (affected area image), information on the size of the affected region 402, a object ID (identification information) of the object 401, a name of the object 401, a date and time of capturing the affected area image and the like, and stores the records in the image processing apparatus 3.

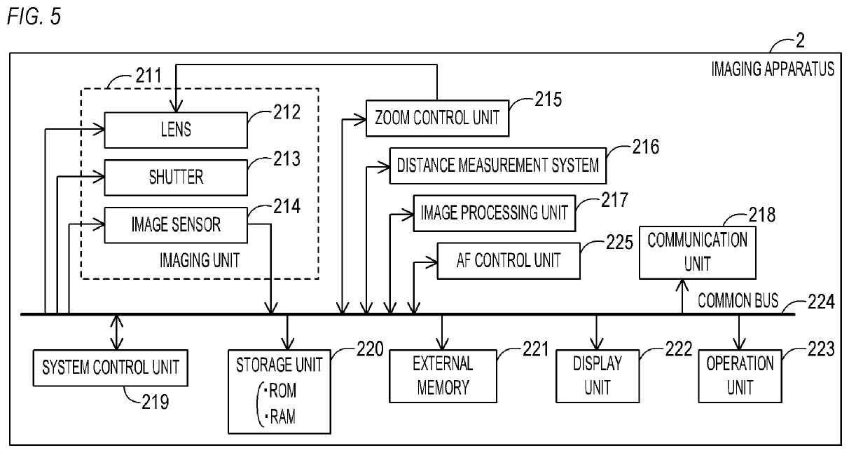

FIG. 5 is an example of a hardware configuration of the imaging apparatus 2. The imaging apparatus 2 is a camera which includes an AF control unit 225, an imaging unit 211, a zoom control unit 215, a distance measurement system 216, an image processing unit 217, a communication unit 218, a system control unit 219, a storage unit 220, an external memory 221, a display unit 222, an operation unit 223 and a common bus 224.

The AF control unit 225 extracts high frequency components of the imaging signal (video signal), searches a lens position where the high frequency component is at the maximum (position of a focus lens included in the lens 212), and controls the focus lens, whereby a focal point is automatically adjusted. This focus control system is also called TV-AF or contrast AF, and can implement high precision focusing. Further, the AF control unit 225 acquires a distance to the object based on the focal point adjustment amount or the moving distance of the focus lens, and outputs the acquired distance. The focus control system is not limited to the contrast AF, but may be a phase difference AF or other AF systems. The AF unit 10 in FIG. 3 is implemented by operation of the AF control unit 225.

The imaging unit 211 includes a lens 212, a shutter 213, and an image sensor 214. The imaging unit 11 (functional unit) of the imaging apparatus 2 in FIG. 3 is implemented by operation of this imaging unit 211. The lens 212 forms an optical image of an object on the image sensor 214. The image sensor 214 is constituted of a charge storage type solid-state image sensor (e.g. CCD, CMOS element) that converts an optical image into electric signals. The imaging unit 211 includes in the lens 212 an aperture that determines an aperture value to adjust an exposure amount. The shutter 213 performs open/close operation to expose or shield the light for the image sensor 214, and controls the shutter speed. The shutter is not limited to a mechanical shutter, but may be an electronic shutter. In the case of an image pickup element using a CMOS sensor, the electronic shutter performs reset scanning to set the stored charge amount of each pixel to zero for each pixel or for each region (e.g. each line) constituted of a plurality of pixels. Then for each pixel or each region for which reset scanning is performed, scanning to read signals is performed after a predetermined time elapses.

The zoom control unit 215 controls the driving of a zoom lens included in the lens 212. The zoom control unit 215 drives the zoom lens via a zoom motor (not illustrated) in accordance with the instructions from the system control unit 219. Thereby zooming is performed.

The distance measurement system 216 is a unit to acquire a distance to the object. The distance measurement system 216 may generate the distance information based on the output of the AF control unit 225. If a plurality of blocks, each of which is constituted of at least one pixel in the screen (display surface) of the display unit 222, are set, the distance measurement system 216 detects a distance for each block by repeatedly moving the AF for each block. For the distance measurement block 216, a system using a time of flight (TOF) sensor may be used. The TOF sensor is a sensor to measure the distance to an object based on the time difference (or phase difference) between the transmitting timing of an emitted wave and a receiving timing of a reflected wave, which is the emitted wave that is reflected by the object. Further, for the distance measurement system 216, a position sensitive device (PSD) system may be used where a PSD is used for each light-receiving element. The information generation unit 13 (functional unit) of the imaging apparatus 2 in FIG. 3 is implemented by operation of the distance measurement system 216.

The image processing unit 217 performs image processing on RAW image data outputted from the image sensor 214. The image processing unit 217 performs various image processing operations, such as white balance adjustment, gamma correction, color interpolation (demosaicing) and filtering, on an image outputted from the imaging unit 211 (RAW imaging data), or an image stored in the later mentioned storage unit 220. The image processing unit 217 also performs compression processing based on such standard as JPEG, on an image captured by the imaging unit 211. The image processing unit 12 (functional unit) of the imaging apparatus 2 in FIG. 3 is implemented by the operation of the image processing unit 217.

The communication unit 218 is a communication interface for each component of the imaging apparatus 2 to communicate with an external apparatus (e.g. image processing apparatus 3) via a wireless network (not illustrated). The output unit 15 and the second acquisition unit 16 (functional units) of the imaging apparatus 2 in FIG. 3 are implemented by the operation of the communication unit 218. A specific example of a network is a network based on the Wi-Fi (R) standard. Communication using Wi-Fi may be implemented via a router. The communication unit 218 may be implemented by a cable communication interface such as USB and LAN.

The system control unit 219 includes a central processing unit (CPU), and controls each unit of the imaging apparatus 2 in accordance with the programs recorded (stored) in the storage unit 220 (general control). For example, the system control unit 219 controls the AF control unit 225, the imaging unit 211, the zoom control unit 215, the distance measurement system 216 and the image processing unit 217,

The storage unit 220 temporarily stores various setting information (e.g. information on focus position when an image is captured) required for operation of the imaging apparatus 2, and various images (e.g. image captured by the imaging unit 211 and image processed by the image processing unit 217). The storage unit 220 may temporarily store image data and analysis data (e.g. information on size of object) received by the communication unit 218 communicating with the image processing apparatus 3. The storage unit 220 is constituted of an erasable non-volatile memory (e.g. flash memory, SDRAM).

The external memory 221 is a non-volatile storage medium that is inserted into or embedded in the imaging apparatus 2, and is an SD card or CF card, for example. This external memory 221 stores, for example, image data processed by the image processing unit 217, and image data and analysis data received by the communication unit 218 communicating with the image processing apparatus 3. The image data, analysis data or the like, recorded in the external memory 221, can be read and outputted outside the imaging apparatus 2.

The display unit 222 displays an image temporarily stored in the storage unit 220, image and information stored in the external memory 221, and a setting screen of the imaging apparatus 2, for example. The display unit 222 is a thin film transistor (TFT) liquid crystal display, an organic EL display, an electronic view finder (EVF) or the like. The display unit 14 (functional unit) of the imaging apparatus 2 in FIG. 3 is implemented by operation of the display unit 222.

The operation unit 223 is a receiving unit to receive a user operation, and includes buttons, switches, keys, mode dial and the like included in the imaging apparatus 2. The operation unit 223 may include a touch panel which is also used for the display unit 222. The instructions for various mode settings and image capturing operations by the user are sent to the system control unit 219 via the operation unit 223.

The above mentioned AF control unit 225, imaging unit 211, zoom control unit 215, distance measurement system 216, image processing unit 217, communication unit 218, system control unit 219, storage unit 220, external memory 221, display unit 222 and operation unit 223 are connected to the common bus 224. The common bus 224 is a signal line to send/receive signals between each block.

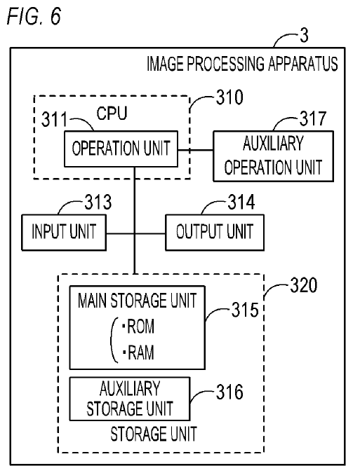

FIG. 6 is an example of a hardware configuration of an information processing apparatus (image processing apparatus 3). The image processing apparatus 3 is a computer which includes a central processing unit (CPU) 310, a storage unit 312, an input unit 313 (e.g. mouse, keyboard), an output unit 314 (e.g. display) and an auxiliary operation unit 317. The CPU 310 includes an operation unit 311. The storage unit 312 includes a main storage unit 315 (e.g. ROM, RAM), and an auxiliary storage unit 316 (e.g. magnetic disk, solid-state drive (SSD)). A part of the input unit 313 and the output unit 314 is constructed as a wireless communication module to perform Wi-Fi communication.

The auxiliary operation unit 317 is an IC for auxiliary operation under the control of the CPU 310. For the auxiliary operation unit 317, a graphic processing unit (GPU), for example, can be used. A GPU is a processor for image processing, and includes a plurality of product-sum operation units, and is often used as a processor to perform processing for signal learning since a GPU excels in matrix calculations. A GPU is also used for processing to perform deep learning. For the auxiliary operation unit 317, a field-programmable gate array (FPGA), an ASIC or the like may be used.

The operation unit 311 included in the CPU 310 functions as the acquisition unit 21, the extraction unit 22, the superimposing unit 23, the analysis unit 24, the second output unit 25, the storage unit 26, the reading unit 30 and the recognition processing unit 31 of the imaging processing apparatus 3 in FIG. 3 by executing the programs recorded (stored) in the storage unit 312. The operation unit 311 also controls the processing execution sequence.

A number of CPUs 310 and a number of storage units 312 of the image processing apparatus 3 may be one or a plurality thereof. In other words, at least one processing unit (CPU) and at least one storage unit are connected to the image processing apparatus 3, and the image processing apparatus 3 may function as each of the abovementioned units if at least one processing unit executes programs recorded in at least one storage unit. The processor is not limited to a CPU, but may be an FPGA, an ASIC or the like.

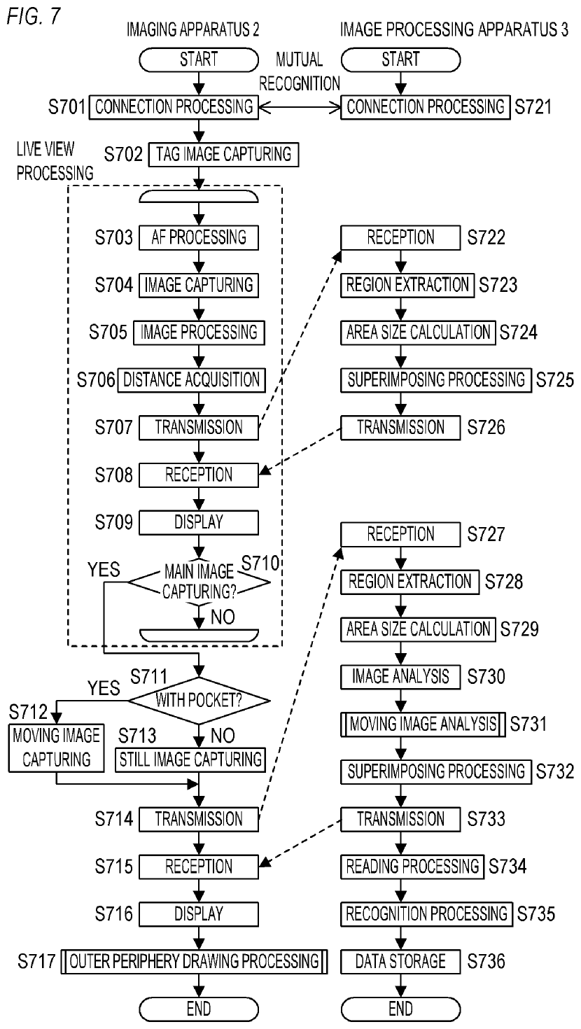

The operation of the image processing system 1 according to Embodiment 1 will be described with reference to the flow chart in FIG. 7. In the flow chart in FIG. 7, the processing of the imaging apparatus 2 is implemented by developing programs, which are recorded in ROM (a part of the storage unit 220), in RAM (a part of the storage unit 220), and the system control unit 219 executing the programs. In the same manner, the processing of the image processing apparatus 3 is implemented by developing programs, which are recorded in ROM (a part of the main storage unit 315), in RAM (a part of the main storage unit 315), and the CPU 310 executing the programs. In the flowchart in FIG. 7, to evaluate the bedsore of the ulcerous surface, one frame of the captured moving image data is analyzed, and the size of the ulcerous surface is measured. Further, a composite image, to detect the size of the pocket, is generated by the image processing apparatus 3, and is sent to the imaging apparatus 2. The processing in FIG. 7 starts when power of the imaging apparatus 2 and power of the image processing apparatus 3 are turned ON, and operation to interconnect the imaging apparatus 2 and the image processing apparatus 3 is performed.

In step S701 and step S721, the imaging apparatus 2 and the image processing apparatus 3 perform connection processing to connect with each other for communication. For example, the system control unit 219 of the imaging apparatus 2 is connected to a Wi-Fi standard (wireless LAN standard) network (not illustrated) using the communication unit 218. The CPU 310 of the image processing apparatus 3 is also connected to the same network using the input unit 313 and the output unit 314. Then in step S721, the CPU 310 performs search processing to search for the imaging apparatus to be connected to, and in S701, the system control unit 219 performs response processing to respond to the search processing. For the search processing, various apparatus search techniques can be used to search (retrieve) an apparatus via the network. For example, a search processing using universal plug and play (UPnP) is performed, and an individual apparatus is identified using the universally unique identifier (UUID).

In step S702, the system control unit 219 of the imaging apparatus 2 captures the image of the barcode tag 403 of the object 401 using the imaging unit 211. The barcode tag 403 includes the object ID (patient ID) that identifies the object 401 (patient). By capturing the image of the affected area after capturing the image of the barcode tag, the image capturing sequence can be managed based on the date and time of image capturing, and images, from the image of the barcode tag to the image just before the next barcode tag, can be identified as images of the same object based on the object ID.

Then using the imaging unit 211 and the display unit 222, the system control unit 219 of the imaging apparatus 2 performs live view processing in which the live image of the object 401 is displayed on the display unit 222. In the live view processing, the imaging apparatus 2 performs the processing operations in steps S703 to S710. As the live view processing is performed, the image processing apparatus 3 performs the processing operations in steps S722 to S726.

In step S703, the system control unit 219 of the imaging apparatus 2 adjusts the focal point using the AF control unit 225, so that the object 401 is focused on (AF processing). Here in the AF processing, it is assumed that the screen of the display unit 222 is divided into a plurality of blocks, and AF is performed on a predetermined block. In concrete terms, the imaging apparatus 2 is set so that the affected region 402 is disposed at the center of the screen, and AF is performed in the block located at the center of the screen. The AF control unit 225 outputs the distance to the AF area (portion that is focused on by AF) of the object 401 based on the adjustment amount of the focal point or the moving distance of the focus lens, and the system control unit 219 acquires this distance.

In step S704, the system control unit 219 of the imaging apparatus 2 captures an image of the affected region 402 of the object 401 using the imaging unit 211.

In step S705, the system control unit 219 of the imaging apparatus 2 develops an image, which was acquired in step S704, using the image processing unit 217, compressed the developed image based on such standard as JPEG and resizes the acquired JPEG image. The image generated in step S705 is sent to the image processing apparatus 3 in step S707 (described later) by wireless communication. The wireless communication takes a longer time as the size of the image to be sent is larger, hence the image size after resizing is selected considering the allowable communication time. The image generated in step S705 becomes a target of the extraction processing to extract an affected region 402 from the image in step S723 (described later). The image size after resizing depends on the processing time of the extraction processing and the extraction accuracy, hence these conditions are also considered when selecting the image size. Further, step S705 is a part of the live view processing, and if the processing time in step S705 is long, the frame rate of the live image decreases, and operability is affected. Therefore, it is preferable to set the size after resizing to be the same or smaller, compared with the case of the image processing (resizing) in actual image capturing (not live view processing). In step S705, resizing is performed to be 720 pixels×540 pixels, 8-bit RGB color, and 1.1 megabyte of data size. The image size, data size, bit depth, color space and the like after resizing are not especially limited.

In step S706, the system control unit 219 of the imaging apparatus 2 generates the distance information on the distance to the object using the distance measurement system 216. In concrete terms, the system control unit 219 generates the distance information based on the distance outputted by the AF control unit 225 in step S703.

In step S707, using the communication unit 218, the system control unit 219 of the imaging apparatus 2 sends (outputs) the image (image data) generated in step S705 and the distance information generated in step S706 to the image processing unit 3. When this information is transmitted for the first time, the system control unit 219 sends the tag information image captured in step S702 to the image processing apparatus 3 only once.

In step S722, using the input unit 313, the CPU 310 of the image processing apparatus 3 receives (acquires) the image (image of the affected region 402) which the imaging apparatus 2 sent in step S707 and the distance information (distance information corresponding to the object (affected region 402) captured in the image). When this information is received for the first time, the CPU 310 receives the tag information image captured in step S703 only once.

In step S723, the CPU 310 of the image processing apparatus 3 extracts the affected region 402 of the object 401 from the image acquired in step S722. Here the region division (region extraction) is performed only for the ulcerative surface that can be extracted by the image analysis. It is assumed that a method of region division performed here is semantic region division based on deep learning. In other words, using a plurality of images of actual bedsore affected areas as teacher data, models of the neural network are taught to the computer for leaming (not illustrated), so as to generate a learned model. Then the CPU 310 infers an area of the bedsore from the input image based on the generated learned model. It is also assumed that a fully convolutional network (FCN), which is a segmentation model using deep learning, is used as the mode of the neural network. The inference of the deep learning is performed using GPU (included in the auxiliary operation unit 317), which excels in parallel execution of the product-sum operation. The inference processing may be executed by an FPGA or an ASIC. The region division may be implemented using other deep learning models. The segmentation method is not limited to the deep learning, but a method using graph cuts, region growth, edge detection, rule division or the like may be used.

In step S724, the CPU 310 of the image processing apparatus 3 converts the image size (size on the image) of the ulcerous surface region extracted in step S723, so as to analyze (acquire) information on the actual size of the ulcerous surface region. The image size of the ulcerous surface region is converted into the actual size based on the information on the angle of view or the pixel size of the image acquired in step S722, and the distance information acquired in step S722.

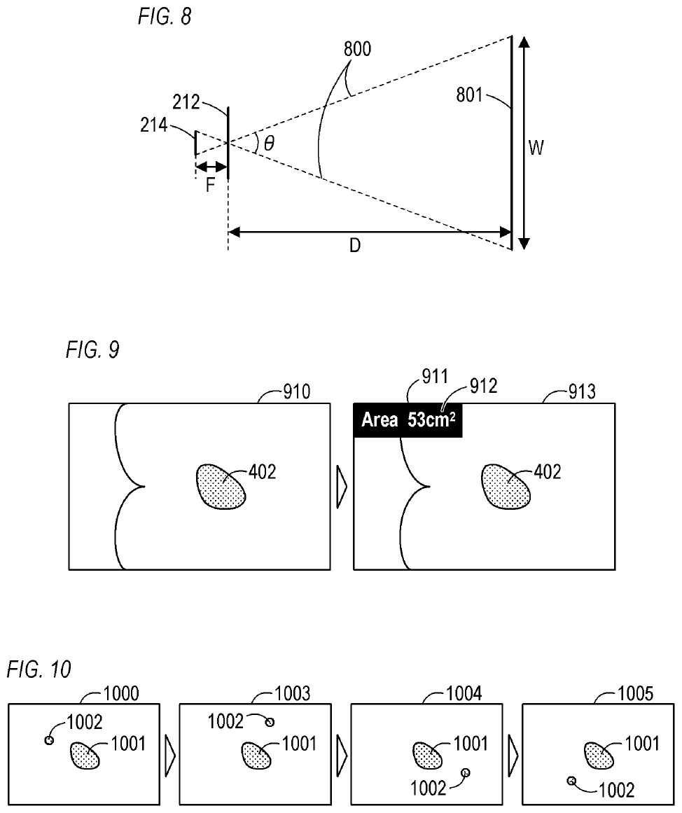

The method of calculating the area size (actual size) of the ulcerous surface region will be described with reference to FIG. 8. A general purpose camera can be handled as a pin hole model illustrated in FIG. 8. The incident light 800 passes through the principal point of the lens 212, and enters the imaging surface of the image sensor 214. The distance from the imaging surface to the principal point of the lens is the focal distance F. In the case of using a thin lens approximation, it is regarded that the two principal points on the front side and the rear side match. Further, in the pin hole model, the lens 212 is regarded as a single lens without thickness, but an actual lens is constituted of a plurality of thick lenses or zoom lens, which include a focus lens. The focal point is adjusted to focus on the object 801 by adjusting the focus lens of the lens 212 so that an image is formed on the imaging surface of the image sensor 214. Furthermore, in the case of the zoom lens, the angle of view θ changes if the focal distance F is changed. In this case, the width W of the object 801 on the focal plane is geometrically determined based on the relationship between the angle of view θ of the imaging apparatus 2 and the object distance D, and the width W of the object 801 can be calculated using a trigonometric function. In other words, the width W of the object 801 is determined by the relationship between the angle of view θ (the parameters are the focus position and zoom amount) and the object distance D. Then the width W of the object 801 is divided by a number of pixels in one line of the image sensor 214, whereby the length on the focal plane corresponding to one pixel of the image is acquired. Further, based on the length on the focal plane corresponding to one pixel, an area size on the focal plane corresponding to one pixel is acquired. The area size of the ulcerous surface region can be calculated by multiplying a number of pixels in the ulcerous surface region extracted in step S723 by the area size on the focal plane corresponding to one pixel.

In step S725, the CPU 310 of the image processing apparatus 3 superimposes the information on the area size (actual size) of the ulcerous surface region (result of processing in step S724) on the image acquired in step S722. The information on the result of extracting the ulcerous surface region may be superimposed.

A state of superimposing information on the area size (actual size) of the ulcerous surface region will be described with reference to FIG. 9. An image 910 in FIG. 9 is an image before the superimposing processing, and includes the ulcerous surface region of the object 401 (affected region 402). The image 913 is an image after the superimposing processing, and a label 911, where a white character string 912 indicating the estimated area size is written on a black background, is superimposed on the image 913 at the upper left corner. For the information on the result of extracting the ulcerous surface region, a frame indicating the ulcerous surface region, for example, is superimposed.

In step S726, the CPU 310 of the image processing apparatus 3 sends (outputs) the information on the actual size of the ulcerous surface region (result of processing in step S724) to the imaging apparatus 2 using the output unit 314. In concrete terms, the CPU 310 outputs the image after the superimposing processing in step S725 (superimposed-processed image) to the imaging apparatus 2 by wireless communication. Information related to the result of extracting the ulcerous surface region may be sent.

In step S708, using the communication unit 218, the system control unit 219 of the imaging apparatus 2 receives (acquires) the information which the image processing apparatus 3 sent in step S726 (superimposed-processed image).

In step S709, the system control unit 219 of the imaging apparatus 2 displays the information received in step S708 (superimposed-processed image) on the display unit 222. Thereby the live view image captured by the imaging unit 211 is displayed, and the information on the actual size of the ulcerous surface region is superimposed and displayed on the live view image. The information may be sent from the image processing apparatus 3 to the imaging apparatus 2, and the superimposing processing may be performed by the imaging apparatus 2, at least as long as either the information on the result of extracting the ulcerous surface region or the information on the actual size of the ulcerous surface region is superimposed and displayed on the live view image.

In step S710, the system control unit 219 of the imaging apparatus 2 determines whether this image capturing operation (operation to instruct this image capturing) is performed on the operation unit 223. If this image capturing operation is performed, live view processing is exited, and processing advances to step S711, and if not, processing returns to step S703 and live view processing is repeated.

In step S711, the system control unit 219 of the imaging apparatus 2 determines whether a pocket exists in the image capturing target bedsore, that is, whether the pocket evaluation using a light, as described with reference to FIG. 2, is necessary. Whether the pocket exists (whether pocket evaluation using the light is required) may be specified by the user (evaluator) using the operation unit 223, or by the system control unit 219 analyzing the live view image. Processing advances to S712 if the pocket exists (if pocket evaluation using the light is required), or to step S713 if not.

In step S712, using the imaging unit 211, the system control unit 219 of the imaging apparatus 2 captures a moving image of a state of the measurement operation using the light (FIG. 2). The system control unit 219 also captures a still image (e.g. still image before the light is inserted into the pocket in the measurement operation using the light). In Embodiment 1, the pocket shape is detected by analyzing the image of the moving path of the light, hence marking using a magic marker or the like is omitted. FIG. 10 is a schematic diagram of each frame of the moving image acquired in step S712. In FIG. 10, a plurality of frames are disposed in a time series, and in the first frame 1000, the ulcerous surface 1001 of the bedsore and the light 1002 emitted from the light are captured. The position of the light 1002 moves as time elapses, in the sequence of the frame 1003, 1004, and then 1005.

In step S713, using the imaging unit 211, the system control unit 219 of the imaging apparatus 2 captures a still image for evaluating a bedsore without a pocket. In concrete terms, AF processing the same as step S703, image capturing the same as step S704, and image processing (e.g. development, resizing) the same as step S705 are performed. Step S713 is not a part of the live view processing, but is a processing of this image capturing processing. Therefore in step S713, priority is assigned to accuracy of measuring the large image size and the bedsore size, rather than a quick processing, and the image is resized to an image size that is the same as or larger than the image size of the image acquired in step S705. Here it is assumed that the image is resized so that the image has 1440 pixels×1080 pixels, 4-bit RGB colors, and a 4.45 megabyte data size. The image size, data size, bit depth, color space and the like after resizing are not especially limited.

In step S714, using the communication unit 218, the system control unit 219 of the imaging apparatus 2 sends (outputs) the image data of the image acquired in this image capturing (moving image and still image captured in step S712 or still image captured in step S713) to the image processing apparatus 3. The system control unit 219 also sends, to the image processing apparatus 3, distance information (object distance) generated in step S706. The distance information may be generated again in this image capturing, so that the distance information generated in this image capturing is sent to the image processing apparatus 3.

In step S727, using the input unit 313, the CPU 310 of the image processing apparatus 3 receives (acquires) the image and the distance information which the imaging apparatus 2 sent in step S714.

In steps S728 to S730, the CPU 310 of the image processing apparatus 3 measures the size of the ulcerous surface of the bedsore. In step S728, just like step S723, the CPU 310 of the image processing apparatus 3 extracts the ulcerous surface region of the object 401 from the image (still image) acquired in step S727. In the case of acquiring a moving image, one frame of the moving image (e.g. one frame before the light is inserted into the pocket in the measurement operation using the light) may be selected, so that the ulcerous surface region is extracted from the selected frame.

In step S729, just like step S724, the CPU 310 of the image processing apparatus 3 analyzes (acquires) the information on the actual size of the ulcerous surface region extracted in step S728 based on the distance information acquired in step S727.

In step S730, the CPU 310 of the image processing apparatus 3 evaluates the ulcerous surface using the image (still image) acquired in step S727. In the case of acquiring the moving image captured in step S712, one frame, out of the plurality of frames of this moving image (e.g. one frame before the light is inserted into the pocket in the measurement operation using the light), may be selected and used.

The evaluation of the ulcerous surface will be described in concrete terms. The CPU 310 of the image processing apparatus 3 analyzes the information on the actual size of the ulcerous surface region, which was extracted in step S728, based on the distance information acquired in step S727, and calculates the major axis, minor axis and the area size of the rectangular region. In the evaluation index of the bedsore determined by DESIGN-R software, it is determined that the size of the bedsore is evaluated by the product of the major axis and minor axis. The image processing system 1 according to Embodiment 1 can acquire the evaluation result that is compatible with the evaluation result conforming to the DESIGN-R software by analyzing the major axis and minor axis. DESIGN-R software does not provide an exact definition for the calculation method, however a plurality of calculation methods are mathematically possible to calculate the major axis and minor axis. For example, among the rectangles circumscribing the ulcerous surface region, a rectangle of which surface region is the smallest (minimum bounding rectangle) is calculated, and the length of the long side and the length of the short side of the minimum bounding rectangle are calculated, so that the length of the long side is regarded as the major axis, and the length of the short side is regarded as the minor axis. The maximum Feret diameter (the maximum caliber length) may be regarded as the major axis, and the length measured in the direction perpendicular to the axis of the maximum Feret diameter may be regarded as the minor axis. For the method of calculating the major axis and the minor axis, an arbitrary method can be selected based on compatibility with the conventional measurement results. The evaluation of the ulcerous surface region is not performed during the live view processing. During the live view processing, it is sufficient if the result of extracting the affected region 402 (ulcerous surface region) can be confirmed, and by omitting the evaluation of the ulcerous surface region, the processing time for the image analysis can be reduced and the frame rate of the live view is increased, whereby the user friendly aspect of the imaging apparatus 2 can be improved.

The processing in step S731 is performed when the moving image (moving image captured in step S712) is acquired in step S727. In step S731, in order to create a composite image to detect the size of the pocket of the bedsore, the CPU 310 of the image processing apparatus 3 analyzes the acquired moving image (image), and acquires various information on this moving image (image). In concrete terms, the information on the locus of the movement of the light is acquired. The method of acquiring information on the moving image is not especially limited, and, for example, the image processing apparatus 3 may acquire the information from an outside source.

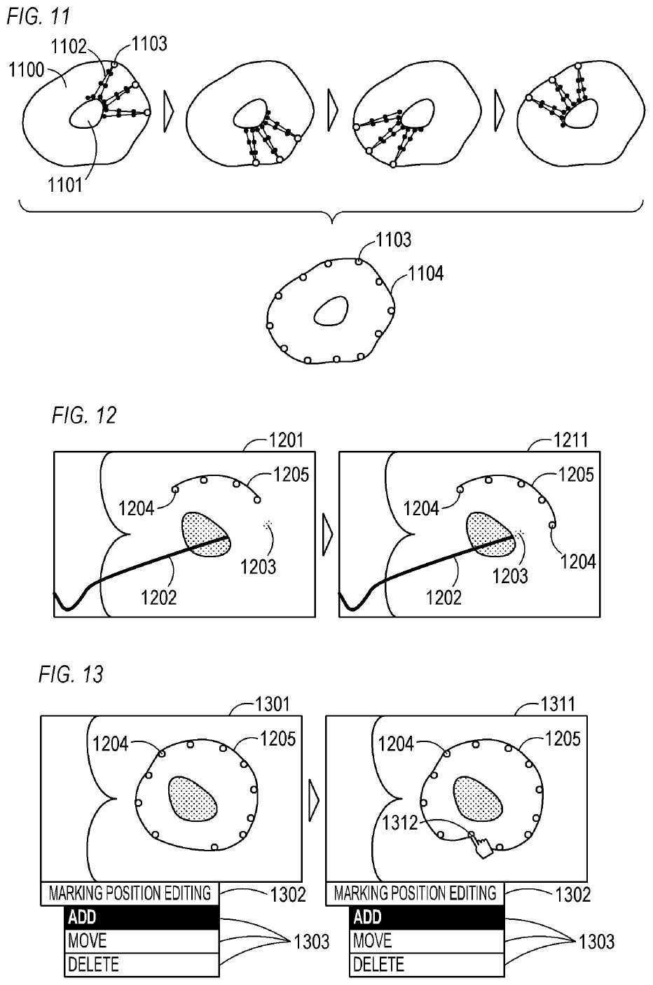

The moving image analysis processing in step S731 executed by the image processing apparatus 3 will be described with reference to FIG. 11. Just like FIG. 2, FIG. 11 indicates a pocket 1100, an ulcerous surface 1101 (entrance portion of the pocket 1100), a path 1102 of the tip of the light, and a point 1103 corresponding to the position of the tip of the light at a point when the tip of the light reached the deepest portion (edge) of the pocket 1100. The pocket 1100 illustrated here is the conceptual surface under the skin, which is actually not visible. The points of the path 1102 indicates a plurality of positions of the tip of the light, which correspond to a plurality of timings respectively.

In the moving image analysis processing in step S731, the CPU 310 detects the position of the tip of the light (point 1103) at the point when the tip of the light reached the deepest portion of the pocket. This point (position) can be regarded as a “point at the edge of the locus of the light moving in the affected area in the diameter direction of the affected area”, or a “position at a boundary between the region of the affected area and a region different from the affected area”. For example, a vertex, when the light moved in the affected area in the diameter direction in the moving image (point where insertion of the light into the pocket changed to the withdrawal of the light), can be detected as the edge point. On the upper side of FIG. 11, an outline of the operation to measure the pocket is indicated in 4 stages in a time series, and in each stage, the point 1103 is detected at 3 locations. On the lower side of FIG. 11, all the detected points 1103 (12 points 1103) are indicated. In the moving image analysis processing, information on the outer periphery of the affected area is acquired based on these points 1103. In concrete terms, the line 1104 combining (connecting) these points 1103, such as a smooth free curve connecting these points 1103 by a spline curve or Bezier curve, is determined (estimated) as the outer periphery of the pocket. Then the pocket shape is determined by analyzing the shape of the acquired line 1104. The information on the outer periphery of the affected area (e.g. shape of outer periphery of affected area, area size of affected area, major axis of affected area, and minor axis of affected area) can be provided to the user by display, or provided to another apparatus as data.

FIG. 12 is an example of live view display during the pocket measurement operation using the light, where a detected marking position (position of the tip of the light when the tip reached the deepest portion of the pocket) and the pocket shape generated (formed) based on the marking positions are displayed.

The screen 1201 is a live view display screen when the tip 1203 of the light 1202 reached the deepest portion of the pocket. As the screen 1201 indicates, the tip 1203 of the light 1202 is emitting light inside the pocket. The position 1204 is a marking position that is acquired by analyzing the movement of the light 1202 in the moving image captured in live view, and the marking position 1204 is displayed at 4 points on the screen 1201. The line 1205 indicates a line (a part of the pocket shape) detected by analyzing the marking position 1204 at these 4 points.

The screen 1211 is a live view display screen when the tip 1203 of the light 1202 is slightly withdrawn from the deepest portion of the pocket after the state of the screen 1201. At this time, this new position of the tip 1203 of the light 1202 on the screen 1201 is acquired as a marking position 1204 by the moving image analysis. By immediately displayed this new marking position 1204 acquired by the moving image analysis on the live view screen, the operator performing the pocket measurement can advance the operation while checking the peripheral shape of the pocket, and whether the pocket measurement operation is being executed correctly. In the case where a new marking position 1204 is displayed by the moving image analysis, the addition of the new marking position 1204 may be notified by blinking the marking position 1204 on screen or by outputting a sound. By performing live view display of the marking position 1204 and the pocket shape 1205 that can be acquired by the moving image analysis during the pocket measurement operation using the light 1202, a desired marking position can be added, or an obviously incorrect marking position can be deleted.

FIG. 13 is an example of the live view display after the pocket measurement operation using the light ends, where the detected marking positions and the pocket shape generated based on the marking positions are displayed. Further, the marking positions can be additionally displayed by an editing operation.

The screen 1301 is a live view display screen when the pocket measurement operation ends (immediately after the pocket measurement operation ended). The marking positions 1204 and the pocket shape 1205 acquired by the moving image analysis are displayed. Further, a marking position edit menu 1302, to edit the marking positions, is displayed adjacent to the screen 1301. The marking position edit menu 1302 includes a plurality of items 1303, where the user can select one of a plurality of items 1303. Here the plurality of items 1303 include “Add”, “Move” and “Delete”. In the screen 1301, “Add” is selected.

In the state where “Add” is selected, the user can add an arbitrary position as a marking position (a position which was not acquired by the moving image analysis). The screen 1311 is a live view display screen when the user selected “Add” and specified a marking position 1312 which is added. As illustrated in the screen 1311, when the user specifies the marking position 1312, this marking position 1312 is additionally displayed. Further, the pocket shape 1205 is updated to a shape generated by analyzing the plurality of marking positions after the addition.

In the state where “Move” is selected, the user can select an arbitrary marking position on the screen and drag and drop the selected marking position, whereby the marking position can be moved. In this case as well, the pocket shape 1205 is updated to the shape generated by analyzing the marking position after the move. In the state where “Delete” is selected, the user can specify (select) an arbitrary marking position on the screen, whereby the specified marking position can be deleted. In this case as well, the pocket shape 1205 is updated to the shape generated by analyzing the remaining marking positions after the delete. In this way, the pocket shape 1205 is updated to a shape connecting the marking positions after the change in accordance with the operation.

Now the description on FIG. 7 continues. In step S732, the CPU 310 of the image processing apparatus 3 superimposes the information on the result of extracting the affected region and information on the size of the affected region on the image (still image) acquired in step S727. In the case of a bedsore with a pocket, not only the information superimposed in step S725 but the result of analyzing the moving image in step S731 is also superimposed. In the case of acquiring the moving image in step S727 (in the case of a bedsore with a pocket), one frame of this moving image (e.g. one frame before the light is inserted into the pocket during the measurement operation using the light) may be selected, so as to superimpose the information on this one frame.

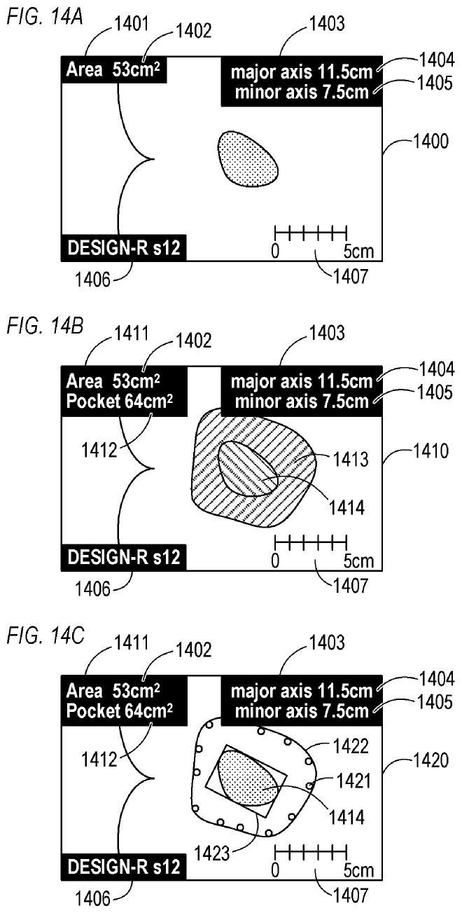

The superimposing processing in step S732 will be described with reference to FIG. 14A to FIG. 14C. Here it is assumed that the information, including the major axis and minor axis, is superimposed as the information indicating the result of extracting the affected region. It is also assumed that information on the marking positions around the pocket, the shape of the pocket and the size of the affected region are superimposed. FIG. 14A to FIG. 14C are examples of a superimposed image (composite image) acquired by the superimposing processing in step S732.

FIG. 14A is an example of a superimposed image in the case of a bedsore without a pocket. In FIG. 14A, a label 1401, where a white character string 1402 indicating the size (the area size) of the ulcerous surface region is written on a black background, is superimposed on a superimposed image 1400 at the upper left corner. Further, a label 1403, where a white character string 1404 indicating the major axis of the ulcerous surface region and a white character string 1405 indicating the minor axis of the ulcerous surface region are written on a black background, is superimposed on the superimposed image 1400 at the upper right corner. Further, a label 1406, where a white character string indicating the index of the size evaluation determined by the DESIGN-R software is written on a black background, is superimposed on the superimposed image 1400 at the lower left corner. Furthermore, a scale bar 1407 is superimposed on the superimposed image 1400 at the lower right corner.

FIG. 14B is an example of a superimposed image in the case of a bedsore with a pocket. In the superimposed image 1410 in FIG. 14B as well, the label 1403, indicating the major axis and the minor axis of the ulcerous surface region, the label 1406 indicating the index of the size evaluation determined by the DESIGN-R software, and the scale bar 1407 are superimposed in the same manner as FIG. 14A. In the superimposed image 1410 in FIG. 14B, however, the label 1411 is superimposed instead of the label 1401 in FIG. 14A. In the label 1411, not only the character string 1402 indicating the area size of the ulcerous surface region, but the character string 1412 indicating the area size of the pocket is also written. The area size of the pocket is also calculated based on the object distance and the like, just like the area size of the ulcerous surface region. Furthermore, in the superimposed image 1410 in FIG. 14B, the pocket region 1413 and the ulcerous surface region 1414 are filled with different colors. By color coding like this, the pocket region 1413 and the ulcerous surface region 1414 can be visually discerned with more accuracy.

In the case of a bedsore without a pocket (in the case of FIG. 14A), a character string indicating that the pocket does not exist (e.g. “Pocket 0”, “No Pocket”) may be superimposed instead of the character string indicating the area size of the pocket (character string 1412 in FIG. 14B). Further, a frame (line) to indicate the contour of the region may be superimposed so that the pocket region and the ulcerous surface region can be visually discerned with more accuracy. In the case of a bedsore without a pocket (in the case of FIG. 14A) as well, the ulcerous surface region may be filled or the frame indicating the contour of the ulcerous surface region may be superimposed. By operating the imaging apparatus, the display of only the pocket region, the display of only the ulcerous surface region, or the display of both the pocket region and the ulcerous surface region may be selected. The image can then be confirmed focusing on only one of the pocket region and the ulcerous surface region.

FIG. 14C is another example of a superimposed image in the case of a bedsore with a pocket. In the superimposed image 1420 in FIG. 14C as well, the label 1411, the label 1403, the label 1406 and the scale bar 1407 are superimposed in the same manner as FIG. 14B. In FIG. 14C, however, the pocket region and the ulcerous surface region are not filled, but a plurality of points 1421 indicating a plurality of marking positions around the pocket and a line 1422 indicating the shape of the pocket are superimposed. Here it is assumed that the major axis and the minor axis were calculated using the minimum bounding rectangle. In the superimposed image 1420 in FIG. 14C, a rectangle frame 1423 indicating the minimum bounding rectangle surrounding the ulcerous surface region 1414 is superimposed. In the case of a bedsore without a pocket (in the case of FIG. 14A) as well, the rectangle frame indicating the minimum bounding rectangle may be superimposed.

Now the description on FIG. 7 continues. In step S733, the CPU 310 of the image processing apparatus 3 sends the composite image (superimposed image) created in step S732 to the imaging apparatus 2 using the output unit 314. The information on the affected region may be sent from the image processing apparatus 3 to the imaging apparatus 2, so that the imaging apparatus 2 creates a composite image.

In step S734, the CPU 310 of the image processing apparatus 3 reads the object ID used for identifying the object, from a one-dimensional barcode (not illustrated) included in the image captured in step S702. The timing of transmitting the image captured in step S702 is not especially limited. For example, the imaging apparatus 2 may output the image captured in step S702 to the image processing apparatus 3 in step S714, and the image processing apparatus 3 may acquire the image captured in step S702 from the imaging apparatus 2 in step S727.

In step S735, the CPU 310 of the image processing apparatus 3 collates the object ID, which was read in step S734, with the object IDs, which were registered in advance, and acquires (determines) the name of the current object. If the name and object ID of the current object are not registered, the CPU 310 prompts the user to register the name and object ID of the current object, and acquires this information.

In step S736, the CPU 310 of the image processing apparatus 3 records the object information, which includes the result of evaluating the affected area (analysis result in step S730 and step S731), in the auxiliary storage unit 316 as the object data determined in step S735. If the data linked to the current object (object ID) is not recorded, the CPU 310 creates new object information, and if the data linked to the current object (object information) is not recorded, the object information is updated.

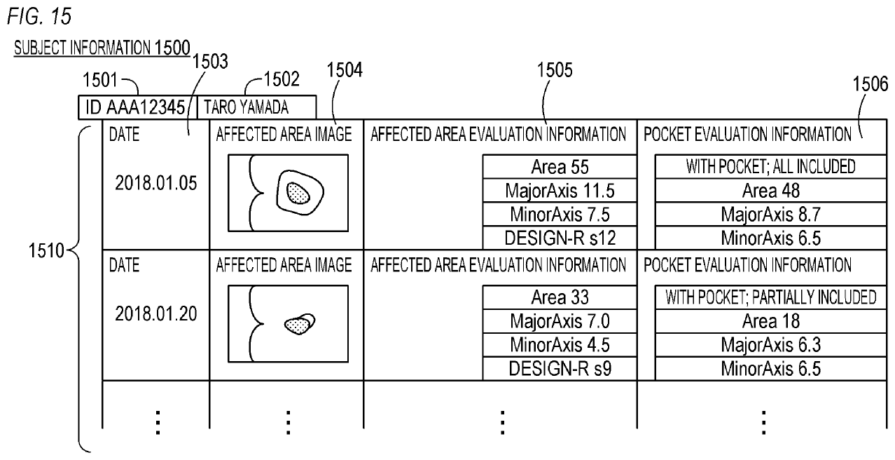

The data configuration of object information 1500 that is stored in the image processing apparatus 3 will be described with reference to FIG. 15. The object information 1500 includes an object ID 1501, a name 1502 of the object, and affected area information 1510 corresponding to the object ID 1501 and the name 1502. The affected area information 1510 is managed for each image capturing data and time. In concrete terms, the patient information 1510 includes at least one combination of the date information 1503, affected area image 1504, affected area evaluation information 1505, and pocket evaluation information 1506. The date information 1503 is information on the date when the affected area was captured, and the affected area image 1504 is an image that was used for evaluating the affected area. The affected area evaluation information 1505 includes a value acquired by evaluating the affected area which includes both the ulcerous surface and the pocket. In the example in FIG. 15, the affected area evaluation information 1505 includes the size of the affected region which includes both the ulcerous surface and the pocket, the major axis of the affected region, the minor axis of the affected region, and the evaluation value determined by the DESIGN-R software. The pocket evaluation information 1506 includes a value acquired by evaluating the pocket. In the example in FIG. 15, the pocket evaluation information 1506 includes the pocket state information indicating the state of the pocket, size of the pocket, major axis of the pocket, and minor axis of the pocket. For example, as the pocket state information, the text information “with pocket, complete inclusion” is registered for a bedsore with a pocket that completely includes the ulcerous surface, “with pocket, partial inclusion” is registered for a bedsore with a pocket which partially overlaps with the ulcerous surface, and “no pocket” is registered for a bedsore without a pocket. The pocket state information may be registered by the user inputting the information, or may be automatically registered by image analysis. In this way, the affected area evaluation information 1505 and the pocket evaluation information 1506 are separately generated (calculated) and recorded. The object information 1500 can be provided to the user by display or the like, or provided to another apparatus as data.

In step S715, using the communication unit 218, the system control unit 219 of the imaging apparatus 2 receives (acquires) the composite image (superimposed image) which the image processing apparatus 3 sent in step S733.

In step S716, the system control unit 219 of the imaging apparatus 2 displays the composite image received in step S715 on the display unit 222.

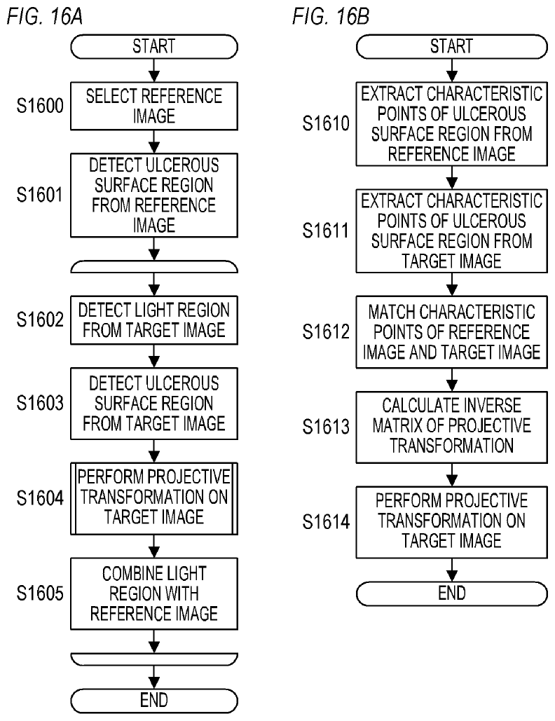

An example of the moving image analysis processing in step S731 in FIG. 7 will be described with reference to the flow chart in FIG. 16A. Here an example of generating the composite image to detect the size of the pocket of the bedsore by moving image analysis processing will be described. In this case, in step S732 in FIG. 7, information may be superimposed on the composite image generated in the flow chart in FIG. 16A.

In step S1600, the CPU 310 of the image processing apparatus 3 selects a reference frame (reference image) out of a plurality of frames of the moving image. In the later mentioned step S1605, a light region (light-emitting region of the light; position of the tip of the light; position where the light is emitted) is combined with this reference image. In the frames while measurement is being performed (FIG. 2) using the light, images of a human hand and the light are captured, and it is preferable that a frame before measurement, where no unnecessary images are captured, is selected as the reference image. For example, a reference image where no unnecessary images are captured can be acquired by starting capturing the moving image before the light is inserted into the pocket, and selecting the first frame of the moving image as the reference image. A frame in which a region corresponding to the light is not included may be selected as the reference image by acquiring the shape of the light and color information in advance, and analyzing whether the region corresponding to the light is included in the frame of the moving image.

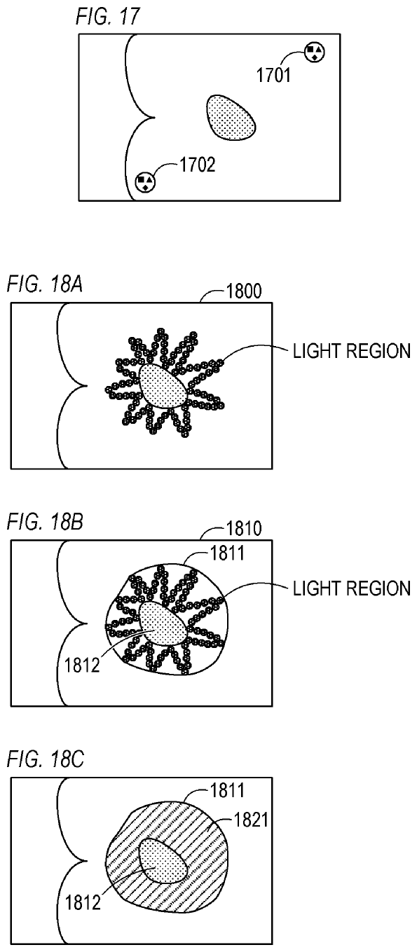

In step S1601, the CPU 310 of the image processing apparatus 3 detects an ulcerous surface region in the reference image. Here the ulcerous surface region is detected to use the result of detecting the ulcerous surface region as a reference to combine the light region. There is no need to use the ulcerous surface region as a reference to combine the light region if the imaging apparatus 2 and the object do not move at all during measurement, but in practical terms this is difficult to do, hence the reference region, such as the ulcerous surface region, is set to combine with the light region. The ulcerous surface region is detected in the same method as step S728 in FIG. 7. During measurement using the light, the ulcerous surface region may be hidden by the light or the hand of the operator. Considering such a case, markers 1701 and 1702 may be disposed near the ulcerous surface, as illustrated in FIG. 17, so that the disposed markers are detected as a reference to combine the light region. In the example in FIG. 17, two markers 1701 and 1702 are disposed considering the case where a marker is hidden during measurement. The number of markers, however, may be 3 or more. If there is a physical characteristic on the body of the patient, this may be used as the reference.

The processing performed in each step S1602 to S1605 to be described next is repeated one frame at a time, such that the processing is performed for all frames of the moving image. In step S1602, the CPU 310 of the image processing apparatus 3 detects the light region in the target image (processing target frame). Here the characteristic of the light region is red and round. In step S1602, a region having this characteristic is detected in the target image as the light region. A red point that is moving in the moving image without changing the predetermined size (change of the size of the red point in the moving image remaining within a predetermined range) may be regarded as the position of the light. In step S1603, the ulcerous surface region is detected in the target image. As mentioned above, the ulcerous surface region must be detected to combine the light region with the ulcerous surface region as a reference. In step S1604, the projective transformation is performed on the target image. During the measurement using the light, the relative direction and position of the imaging apparatus 2, with respect to the object, may change, therefore in order to combine the light region accurately, the projective transformation is performed. A concrete method of the projective transformation will be described later. In step S1605, the light region after the projective transformation is combined with the reference image. By performing the processing steps S1602 to S1605 for all frames, the composite image 1800 in FIG. 18A can be acquired. It is also possible to determine the locus of the light using a frame at every predetermined time to detect the ulcerous surface region. In this case, it is preferable that the image for combining includes frames acquired when the light reached an edge of the affected area, even if these frames are not the frames corresponding to the frame at every predetermined time. For example, the composite image is created by combining the light region using the images of frames at every T=0.01 seconds, 0.02 seconds or 0.03 seconds. At this time, it is preferable that the position of the light with respect to the affected area is detected in each image, and an item that indicates the position of the light is displayed. In other words, only the light of the reference image is displayed as an actual light, and each light in the other images is displayed as a red dot or black dot, for example, at a position corresponding to the light position in the reference image.

The light at an edge position of the affected area in the diameter direction may be displayed in a display format that is different from the light at the other positions, so that the points on the edge can be clearly seen. For example, the brightness of the light at the edge position may be increased when the composite image is generated. Further, in the case of indicating a position of the light by an item, the color of the item at the edge may be changed in the display. A line or the like to indicate the locus of the light may be displayed. In this way, the user can easily draw the outer periphery of the ulcerous region by clearly recognizing the edge position and locus of the light.

The image for the composition may be acquired at each time the light moves a predetermined distance, not at every predetermined time.

A still image captured with the moving image may be used as the reference image, or the processing result in step S728 may be used instead of the processing result in step S1601.

The processing in step S1604 (projective transformation) in FIG. 16A will be described next with reference to the flow chart in FIG. 16B. In step S1610, the CPU 310 of the image processing apparatus 3 extracts the characteristic points of the ulcerous surface region of the reference image (ulcerous surface region detected in step S1601). Here it is assumed that arbitrary points on the outer periphery of the ulcerous surface region are extracted as the characteristic points. In step S1611, the CPU 310 of the image processing apparatus 3 extracts the characteristic points from the ulcerous surface region of the target image, just like step S1610. In step S1612, the CPU 310 of the image processing apparatus 3 matches the characteristic points extracted in step S1610 (characteristic points in the ulcerous surface region of the reference image), and the characteristic points extracted in step S1611 (characteristic points in the ulcerous surface region of the target image). By this matching, the corresponding characteristic points between the reference image and the target image are identified. In step S1613, the CPU 310 of the image processing apparatus 3 calculates, based on the matching result in step S1612, the inverse matrix of the projective transformation so that the ulcerous surface region of the target image becomes the same region (plane) as the ulcerous surface region of the reference image. In step S1614, the CPU 310 of the image processing apparatus 3 performs the projective transformation of the target image using the inverse matrix calculated in step S1613. By performing this projective transformation, an image, of which change of the direction of the imaging apparatus 2 with respect to the object is suppressed, can be acquired.

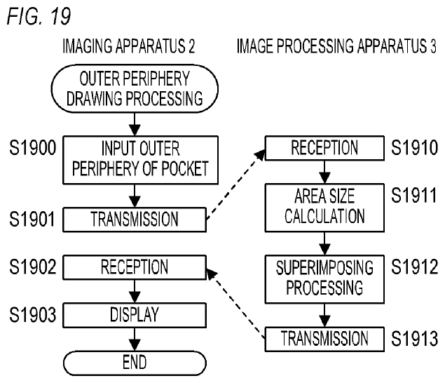

The composite image 1800 in FIG. 18A can be received transmitted in steps S733 and S715 in FIG. 7, and displayed on the imaging apparatus 2 in step S716 (providing the composite image). In this case, in step S717 in FIG. 7, the system control unit 219 performs an outer periphery drawing processing to draw the outer periphery of the pocket. The outer periphery drawing processing after the composite image 1800 is displayed on the imaging apparatus 2 will be described with reference to the flow chart in FIG. 19. In the composite image 1800, the locus of the movement of the light can be visually recognized, hence the user can easily identify the region of the pocket. The method of providing the composite image is not especially limited, as long as the information on the locus of the movement of the light is provided.

In step S1900, the system control unit 219 of the imaging apparatus 2 prompts the user to input the output periphery of the pocket. The output periphery of the pocket may be inputted by the user tracing the outer periphery on the screen of the imaging apparatus 2 (display unit 222) using a finger, or may be inputted by using such an input device as a touch pen. FIG. 18B is a display example after the user inputted the outer periphery of the pocket. In the image 1810 in FIG. 18B, the outer periphery 1811 of the pocket is inputted along the vertexes (outer side) of the light region, and the outer periphery 1811 of the pocket is superimposed and displayed on the composite image 1800 in FIG. 18A.

In step S1901, using the communication unit 218, the system control unit 219 of the imaging apparatus 2 sends the composite image 1810 on which the outer periphery 1811 of the pocket is drawn, and pocket outer periphery information on the outer periphery 1811 of the pocket, to the image processing apparatus 3.

In step S1910, using the input unit 313, the CPU 310 of the image processing apparatus 3 receives the composite image 1810 and the pocket outer periphery information which the imaging apparatus 2 sent in step S1901.

In step S1911, the CPU 310 of the image processing apparatus 3 calculates the area size (size) of the pocket region based on the composite image 1810 and the pocket outer periphery information received in step S1910. Here it is assumed that the area size of the pocket region is calculated by subtracting the area size of the ulcerous surface region 1812 from the area size of the region surrounded by the outer periphery 1811 of the pocket. In other words, the area size of the portion of the region 1821 in FIG. 18C is calculated. The area size may be calculated in accordance with the calculation method of the DESIGN-R software.

In step S1912, the CPU 310 of the image processing apparatus 3 superimposes information on the pocket region and the area size thereof (calculated in step S1911) on the reference image (image based on which the composite image 1800 is generated). Thereby the composite images illustrated in FIG. 14B and FIG. 14C are acquired.

In step S1913, using the output unit 314, the CPU 310 of the image processing apparatus 3 sends the composite image created in step S1912 to the imaging apparatus 2.

In step S1902, using the communication unit 218, the system control unit 219 of the imaging apparatus 2 receives the composite image which the image processing apparatus 3 sent in step S1913.

In step S1903, the system control unit 219 of the imaging apparatus 2 displays the composite image received in step S902. Thereby the size of the pocket region can be measured without drawing the pocket region directly on the skin of the patient (object) using a magic marker.

In FIG. 16A and FIG. 16B, a composite image in which the positions of the light region are accurately reflected is created by combining the light region after performing the projective transformation. Another method is using a focal distance when the image is captured. The distance between the patient and the imaging apparatus 2 may be changed during the image capturing (measurement) since it is time consuming to measure the pocket region using a light. Here the focal distance information can also be acquired during image capturing, hence the image can be magnified or demagnified using this information.

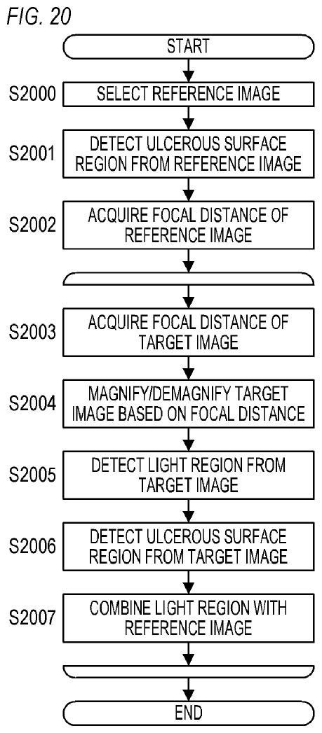

A method of creating a composite image using the focal distance when an image is captured will be described with reference to the flow chart in FIG. 20. Step S2000 is the same as step S1600 in FIG. 16A, and step S2001 is the same as step S1601 in FIG. 16A. In step S2002, the CPU 310 of the image processing apparatus 3 acquires the focal distance of the reference image. The processing steps S2003 to S2007 are repeated for one frame at a time, so as to be performed for all the frames of the moving image. In step S2003, the CPU 310 of the image processing apparatus 3 acquires the focal distance of the target image. In step S2004, the CPU 310 of the image processing apparatus 3 magnifies or demagnifies the target image, so as to match with the focal distance of the reference image. Step S2005 is the same as step S1602 in FIG. 16A, step S2006 is the same as step S1603, and step S2007 is the same as step S1605. By using the focal distance like this, a composite image, in which the position of the light region is accurately reflected, can be created.

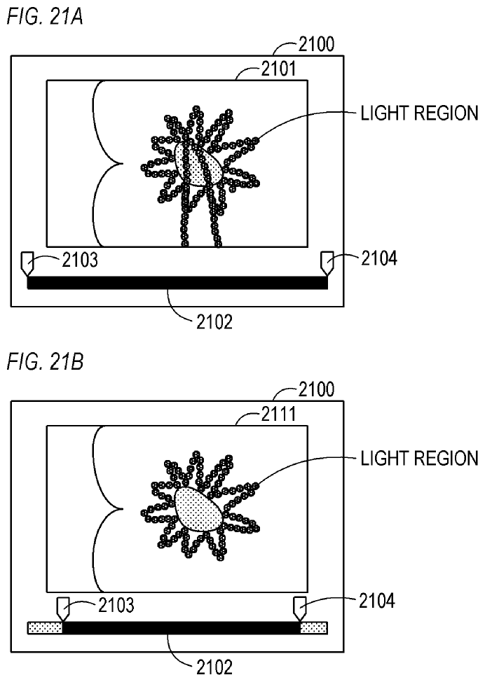

20 In FIG. 16A, FIG. 16B and FIG. 20, if all the frames of the captured moving image are used as the target images, the light region of the frames before and after inserting the light into the pocket may be combined. If the composite image acquired like this is used, the locus of the light region is difficult to identify. Therefore operability improves if the frames (light regions) in a specified period can be deleted. FIG. 21A and FIG. 21B indicate a UI (screen 2100) on which such an operation can be performed.

The screen 2100 includes the control items 2102 to 2104 to delete unnecessary frames (unnecessary light regions) from the composite image. The item 2102 is a slide bar which indicates the time axis, and the items 2103 and 2104 are sliders to delete the unnecessary frames. The unnecessary frames can be deleted by moving the sliders 2103 and 2104 to the left or right. In the state in FIG. 21A, the sliders 2103 and 2104 are disposed on each end of the slider bar 2102, and a composite image 2101 generated by combining all the frames of the moving image is displayed. In the composite image 2101, frames (light regions) before and after inserting the light into the pocket are also combined, which makes the locus of the light region difficult to identify. In the state in FIG. 21B, on the other hand, the range from the slider 2103 to slider 2104 is decreased compared with FIG. 21A. In this case, the frames before the frame corresponding to the slider 2103 and the frames after the frame corresponding to the slider 2104 are not combined. As a result, the composite image 2111, in which the frames before and after inserting the light into the pocket are not combined and the locus of the light region can be easily identified, can be displayed. By adjusting the positions of the sliders 2103 and 2104 like this, the light region in the unnecessary period can be removed from the combining targets.

According to Embodiment 1, the imaging apparatus 2 captures the moving image of the pocket measurement operation using the light, and the image processing apparatus 3 analyzes the moving image and creates the composite image in which the shape of the pocket can be easily identified. Further, by sending this composite image to the imaging apparatus 2, the user can easily specify the pocket region.

### Embodiment 2

In Embodiment 1, the imaging apparatus 2 and the imaging processing apparatus 3 are different apparatuses, but the functional configuration of the image processing apparatus 3 may be included in the imaging apparatus 2 (the imaging apparatus 2 and the image processing apparatus 3 may be integrated). Then such processing as communication between the imaging apparatus 2 and the image processing apparatus 3 becomes unnecessary, and the processing load can be decreased. Further, in Embodiment 1, the composite image in which the pocket region is identified is sent to the imaging apparatus 2, and the user inputs the outer periphery of the pocket to the imaging apparatus 2, but it is not always necessary to input the outer periphery of the pocket to the imaging apparatus 2. For example, the composite image may be stored in the image processing apparatus 3, and an input/output device (e.g. display, mouse) may be connected to the image processing apparatus 3 so that the user can input the outer periphery of the pocket to the image processing apparatus 3. Further, the composite image may be stored in the image processing apparatus 3 in advance, and the user may input the outer periphery of the pocket to an image processing apparatus (e.g. PC, smartphone, tablet) that is different from the image processing apparatus 3, so that the outer periphery of the pocket is notified from this other image processing apparatus to the image processing apparatus 3.

### Embodiment 3

In Embodiment 1, calculation of the area size of the ulcerous surface region and creation of the composite image to detect the size of the pocket region, are executed at the same timing (same flow chart), but these operations may be executed at different timings. For example, depending on the situation at a hospital, measurement of the ulcerous surface region and measurement of the pocket region using the light may be executed at different timings. It is assumed that in such a state, the ulcerous surface region and the pocket region (filled image) are superimposed, as indicated in the superimposed image 1410 (composite image) in FIG. 14B. In this case, the distance between the imaging apparatus 2 and the patient may change between the timing of measuring the ulcerous surface region and the timing of measuring the pocket region, because the posture of the patient changes considerably during measurement, for example. If this occurs, the ulcerous surface region or the pocket region cannot be superimposed at the correct size. In such a case, one of the images (regions) is magnified or demagnified using the focal distance during image capturing, then a composite image, generated by superimposing the ulcerous surface region and the pocket region at accurate sizes, can be acquired.

Even if the measurement of the ulcerous surface region and the measurement of the pocket region using the light are performed at different timings, the ulcerous surface region and the pocket region can easily be superimposed if the image capturing distance does not change between these two timings. For example, in the case where the ulcerous surface region is measured first and the pocket region is measured on another day, the scale of the ulcerous surface region and that of the pocket region become the same if the measurement is performed within the same image capturing distance, and as a result, the images (of the ulcerous surface region and the pocket region) can easily be superimposed. Therefore it is preferable that the image capturing distance during the measurement of the ulcerous surface region is stored, and when the image of the pocket region is captured, the image capturing is started at the timing when the image capturing distance becomes the same as the image capturing distance during the measurement of the ulcerous surface region (at which the ulcerous surface region was imaged for measurement). Once the image capturing is started, the operator must start the measurement of the pocket region using the light, hence the start of the image capturing may be notified to the imaging apparatus 2. By automatically determining the timing of the start of image capturing, the image capturing distance can be made to be consistent among a plurality of measurements.

According to this disclosure, operability can be improved when the affected area (e.g. pocket of bedsore) is measured.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-175565, filed on Sep. 26, 2019, and Japanese Patent Application No. 2019-175334, filed on Sep. 26, 2019, which are hereby incorporated by reference herein in their entirety.

What is claimed is:

1. An image processing system comprising at least one memory and at least one processor which function as:
an acquiring unit configured to acquire information on a captured moving image;
a detecting unit configured to detect, on a basis of the information acquired by the acquiring unit, an edge point of an affected area in a diameter direction thereof from a locus of light moving inside the affected area; and
a providing unit configured to provide information on an outer periphery of the affected area on a basis of a plurality of points detected by the detecting unit.

2. The image processing system according to claim 1, wherein the acquiring unit acquires information on the locus of movement of the light.

3. The image processing system according to claim 1, wherein the detecting unit detects, as the edge point, a vertex when the light moves in the affected area in the diameter direction in the moving image.

4. The image processing system according to claim 1, wherein the edge point is a position at a boundary between a region of the affected area and a region that differs from the affected area.

5. The image processing system according to claim 1,
wherein the light repeats movement in which the light moves inside the affected area in the diameter direction from a predetermined region near a center of the affected area to a position at an edge of the affected area, and then moves to the predetermined region.

6. The image processing system according to claim 1,
wherein the information on the outer periphery of the affected area includes a shape of the outer periphery of the affected area formed by connecting the plurality of points.

7. The image processing system according to claim 1, wherein the information on the outer periphery of the affected area includes an area size of the affected area formed by connecting the plurality of points.

8. The image processing system according to claim 1, wherein the information on the outer periphery of the affected area includes a length of a major axis or a minor axis of the affected area formed by connecting the plurality of points.

9. The image processing system according to claim 1, wherein the plurality of points are connected by a spline curve or a Bezier curve.

10. The image processing system according to claim 1, wherein the providing unit performs control so that the plurality of points and a line connecting the plurality of points are superimposed and displayed on an image of the affected area.

11. The image processing system according to claim 1,
wherein the providing unit performs control so that the moving image is displayed as live view, and a line connecting a plurality of points, which are detected by the detecting unit up to present, is superimposed and displayed on the moving image.

12. The image processing system according to claim 1, wherein
the at least one memory and at least one processor which further function as a receiving unit configured to be capable of receiving an operation to instruct deletion of a point for forming the outer periphery, movement of a point for forming the outer periphery, or addition of a point for forming the outer periphery, and
the providing unit updates the information on the outer periphery on a basis of a plurality of points after change in accordance with the operation.

13. The image processing system according to claim 1,
wherein the providing unit calculates a size of a portion, which is unexposed to outside, of the affected area, as at least a part of the information on the outer periphery on a basis of the affected area formed by connecting the plurality of points and a portion, which is exposed to the outside, of the affected area.

14. The image processing system according to claim 13,
wherein the providing unit calculates the size of the portion which is unexposed to the outside, by a calculation method of DESIGN-R on a basis of the affected area formed by connecting the plurality of points and the portion which is exposed to the outside.

15. The image processing system according to claim 13,
wherein the providing unit further calculates, as a part of the information on the outer periphery, a size of the affected area including the portion which is exposed to the outside and the portion which is unexposed to the outside, and
the image processing system records the size of the affected area and the size of the portion which is unexposed to the outside, separately.

16. The image processing system according to claim 1, wherein the acquiring unit, the detecting unit and the providing unit are included in an imaging apparatus or an information processing apparatus.

17. The image processing system according to claim 1, further comprising a control unit configured to perform control so that capturing of the moving image is automatically started at a timing at which a focal distance becomes same as a focal distance during capturing for measuring a portion, which is exposed to outside, of the affected area.

18. A control method of an image processing system, comprising:
an acquiring step of acquiring information on a captured moving image;
a detecting step of detecting, on a basis of the information acquired in the acquiring step, an edge point of an affected area in a diameter direction thereof from a locus of light moving inside the affected area; and
a providing step of providing information on an outer periphery of the affected area on a basis of a plurality of points detected in the detecting step.

19. A non-transitory computer readable medium that stores a program, wherein the program causes a computer to execute a control method of an image processing system, comprising:
an acquiring step of acquiring information on a captured moving image;
a detecting step of detecting, on a basis of the information acquired in the acquiring step, an edge point of an affected area in a diameter direction thereof from a locus of light moving inside the affected area; and
a providing step of providing information on an outer periphery of the affected area on a basis of a plurality of points detected in the detecting step.

20. An image processing system comprising at least one memory and at least one processor which function as:
an acquiring unit configured to acquire information on a captured image;
a detecting unit configured to detect, on a basis of the information acquired by the acquiring unit, a position of light moving inside an affected area; and
a providing unit configured to acquire information on a locus of movement of the light on a basis of a plurality of positions of the light detected by the detecting unit in a region that is not a portion, which is exposed to outside, of the affected area, and provide recognizably to a user a portion, which is exposed to the outside, of the affected area, and the locus.

21. The image processing system according to claim 20, wherein the image includes the affected area and the light inside the affected area.

22. The image processing system according to claim 20, wherein the acquiring unit acquires information on an image captured at each time the light moves a predetermined distance, or an image captured at each predetermined time.

23. The image processing system according to claim 20, wherein the providing unit performs control so that the locus of the movement of the light and the plurality of positions are superimposed and displayed on the affected area.

24. The image processing system according to claim 20,
wherein the light repeats movement in which the light moves inside the affected area in the diameter direction from a predetermined region near a center of the affected area to a position at an edge of the affected area, and then moves to the predetermined region.

25. The image processing system according to claim 20, wherein the image is one frame of a moving image, or a still image that is captured separately from the moving image.

26. The image processing system according to claim 20,
wherein the acquiring unit further acquires information on a focal distance when the image is captured, and
the providing unit performs control so that the plurality of positions are superimposed and displayed on the affected areas on a basis of the information on the focal distance acquired by the acquiring unit.

27. The image processing system according to claim 20, wherein the providing unit performs control so that the plurality of positions are superimposed and displayed on an image, which is not includes the light, of the affected area.

28. The image processing system according to claim 20, wherein the acquiring unit, the detecting unit and the providing unit are included in an imaging apparatus or an information processing apparatus.

29. The image processing system according to claim 20, wherein at least one memory and at least one processor which function as a calculating unit configured to calculate a size of a portion, which is unexposed to the outside, of the affected area on a basis of the outer periphery of the affected area specified by the user and a portion, which is exposed to the outside, of the affected area.

30. The image processing system according to claim 29, wherein the calculating unit calculates the size of the portion, which is unexposed to the outside, by a calculation method of DESIGN-R on a basis of a portion surrounded by the specified outer periphery and the portion which is exposed to the outside.

31. The image processing system according to claim 29, wherein the providing unit further performs at least one of a first control to display the portion, which is exposed to the outside, by a first color, and a second control to display the portion, which is unexposed to the outside, up to the specified outer periphery, by a second color.

32. The image processing system according to claim 31,
wherein the providing unit performs both the first control and the second control, and
the second color differs from the first color.

33. The image processing system according to claim 31, wherein the providing unit switches execution of the first control and the second control.

34. The image processing system according to claim 20, further comprising a control unit configured to perform control so that capturing of the image is automatically started at a timing at which a focal distance becomes same as a focal distance during capturing for measuring a portion, which is exposed to outside, of the affected area.

35. The image processing system according to claim 20, wherein the providing unit provides, as the information on the locus of the movement of the light, information on a locus excluding a locus in a period specified by the user.

36. A control method of an image processing system, comprising:
an acquiring step of acquiring information on a captured image;
a detecting step of detecting, on a basis of the information acquired in the acquiring step, a position of light moving inside an affected area; and
a providing step of acquiring information on a locus of movement of the light on a basis of a plurality of positions of the light detected in the detecting step in a region that is not a portion, which is exposed to outside, of the affected area, and providing recognizably to a user a portion, which is exposed to the outside, of the affected area, and the locus.

37. A non-transitory computer readable medium that stores a program, wherein the program causes a computer to execute a control method of an image processing system, comprising:
an acquiring step of acquiring information on a captured image;
a detecting step of detecting, on a basis of the information acquired in the acquiring step, a position of light moving inside an affected area; and
a providing step of acquiring information on a locus of movement of the light on a basis of a plurality of positions of the light detected in the detecting step in a region that is not a portion, which is exposed to outside, of the affected area, and providing recognizably to a user a portion, which is exposed to the outside, of the affected area, and the locus.
